(19) United States
(12) Patent Application Publication
(10) Pub. No.: US 2020/0129685 A1
(43) Pub. Date: Apr. 30, 2020

(54) INTRA-AORTIC SPIRAL BALLOON PUMP

(71) Applicant: Cardiac Assist Holding, LLC, Wilmington, DE (US)

(72) Inventors: Barry N. Gellman, Wilmington, DE (US); Kurt A. Dasse, Wilmington, DE (US); Allen B. Kantrowitz, Wilmington, DE (US)

(73) Assignee: Cardiac Assist Holding, LLC, Wilmington, DE (US)

(21) Appl. No.: 16/616,228

(22) PCT Filed: May 23, 2018
(86) PCT No.: PCT/US2018/034051

§ 371 Date: Nov. 22, 2019

Related U.S. Application Data
(60) Provisional application No. 62/510,561, filed on May 24, 2017.

(51) Int. Cl. A61M 1/10; A61M 1/12

(52) U.S. Cl. CPC A61M 1/1072; A61M 2205/0238; A61M 1/122; A61M 1/1001

(57) ABSTRACT

An intra-aortic balloon pump (IABP) is provided that has a series of spiral pleats that function to control the wash of blood associated with an inflation cycle. Additionally, the torsional expansion increases the net efficiency of the IABP relative to a conventional cylindrically shaped balloon. The inflating membrane is textured to promote natural growth of a biologic lining on the surface of the indwelling pump to reduce the need for anticoagulation and the risk of thromboembolic events; promote washing of the surface to minimize stasis and thrombus formation; minimize strain on the IABP; minimize elongation radially and longitudinally to avoid fatigue of the IABP; minimize stretching and stress distribution along a balloon; promote a sweeping effect through the channels in the non-expanded state to wash the surface; or a combination thereof.

### RELATED APPLICATIONS

This application claims priority benefit of U.S. Provisional Application Ser. No. 62/510,561 filed 24 May 2017; the contents of which are hereby incorporated by reference.

### FIELD OF THE INVENTION

The present invention in general relates to cardiac assist devices, and in particular to a spiral balloon pump that is inserted into the aorta of a patient to enhance cardiac output.

### BACKGROUND OF THE INVENTION

Temporary intra-aortic balloon pumps are generally known for insertion through the femoral artery of the leg for emergency patient treatment. Temporary use of the pump was originally intended to last for only a few hours up to a few days for non-ambulatory patients in emergency situations. The temporary intra-aortic balloon pump is limited in size to prevent fully occluding the lumen of the aorta and/or any branch arteries, so that pressures within each location are free to equalize at all times, and in order to pass percutaneously via an introduction sheath through the smaller diameter of the femoral artery during insertion. Non-ambulatory patients restricted to bed can subsist with the level of cardiac assistance available from the relatively small (e.g., typically 30 to 40 cubic centimeters (cc)) volume of the temporary intra-aortic balloon pump. However, this relatively limited level of cardiac assistance is insufficient, and the typical location of insertion is undesirable, for ambulatory patients. In addition, the temporary intra-aortic balloon pump is typically tightly furled and wrapped in order to allow for insertion through a narrow introduction sheath. The furling and wrapping of the material raises concerns regarding damage to the material of the balloon pump which might possibly lead to premature failure when subjected to numerous pumping cycles, if prolonged use over a period greater than a few days is mandated for a particular patient. Further, the power supply conduit to the pump is of limited cross sectional area further limiting the success of such devices.

Subsequent generations of intra-aortic balloon pumps (IABPs) increased the effective pumping volumes of such devices, yet problems have persisted in terms of controlling the wash of blood over the balloon for efficient operation.

Thus, there exists a need for an IABP that addresses these limitations of the prior art.

### SUMMARY

An intra-aortic spiral balloon pump includes a shaft having at least one aperture, and an inflatable membrane encompassing the at least one aperture. The inflatable membrane further includes a plurality of spiral pleats about the shaft.

A cardiac assist device includes an intra-aortic spiral balloon pump as described above, a drive line in fluid communication with the pump; and an external drive unit or fluid supply in fluid communication with the drive line.

### BRIEF DESCRIPTION OF THE DRAWINGS

The subject matter that is regarded as the invention is particularly pointed out and distinctly claimed in the claims at the conclusion of the specification. The foregoing and other objects, features, and advantages of the invention are apparent from the following detailed description taken in conjunction with the accompanying drawings in which:

FIG. 1A is cross sectional view of an inventive balloon in a deflated state along lines A-A of FIG. 1B;

FIG. 1B is a side view of an inventive balloon in deflated form;

FIG. 1C is cross sectional view of an inventive balloon in an inflated state along lines B-B of FIG. 1D;

FIG. 1D is a side view of an inventive balloon in an inflated state;

FIG. 2A is a perspective view of an inventive balloon in deflated form;

FIG. 2B is a perspective view of an inventive balloon in deflated form in a semitransparent form;

FIG. 2C is a perspective view of an inventive balloon in inflated form;

FIG. 2D is a perspective view of an inventive balloon in inflated form in a semitransparent form; and

FIG. 3 is a semitransparent view showing an inventive balloon in a use setting with an external drive system.

### DESCRIPTION OF THE INVENTION

The present invention has utility as an intra-aortic balloon pump (IABP) that has a series of spiral pleats that function to control the wash of blood associated with an inflation cycle. Additionally, the torsional expansion increases the net efficiency of an inventive IABP relative to a conventional cylindrically shaped balloon. In still other inventive embodiments, the inflating membrane is textured to promote natural growth of a biologic lining on the surface of the in-dwelling pump to reduce the need for anticoagulation and the risk of thromboembolic events; promote washing of the surface to minimize stasis and thrombus formation; minimize strain on the IABP; minimize elongation radially and longitudinally to avoid fatigue of the IABP; minimize stretching and stress distribution along a balloon embodiment; promote a sweeping effect through the channels in the non-expanded state to wash the surface; or a combination thereof.

It is to be understood that in instances where a range of values are provided that the range is intended to encompass not only the end point values of the range but also intermediate values of the range as explicitly being included within the range and varying by the last significant figure of the range. By way of example, a recited range of from 1 to 4 is intended to include 1-2, 1-3, 2-4, 3-4, and 1-4.

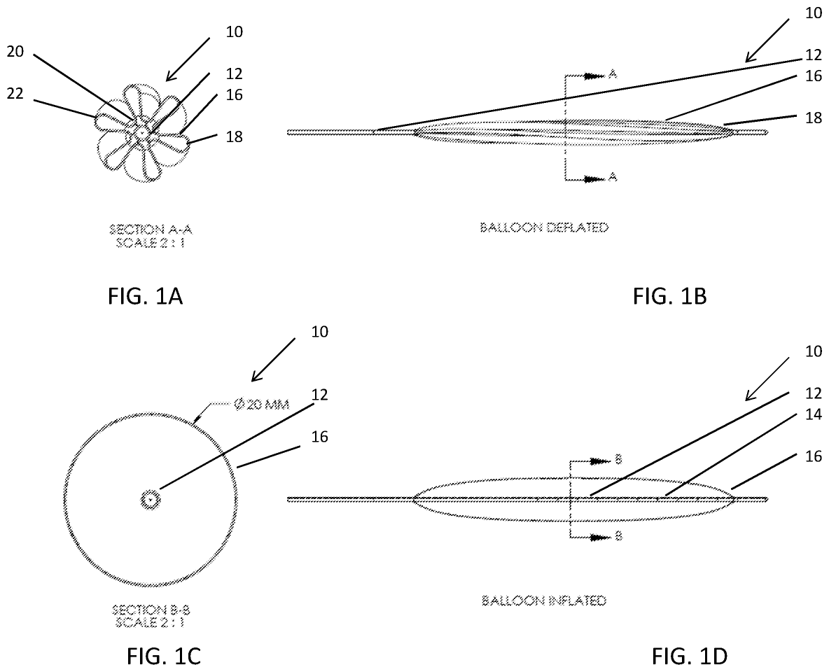

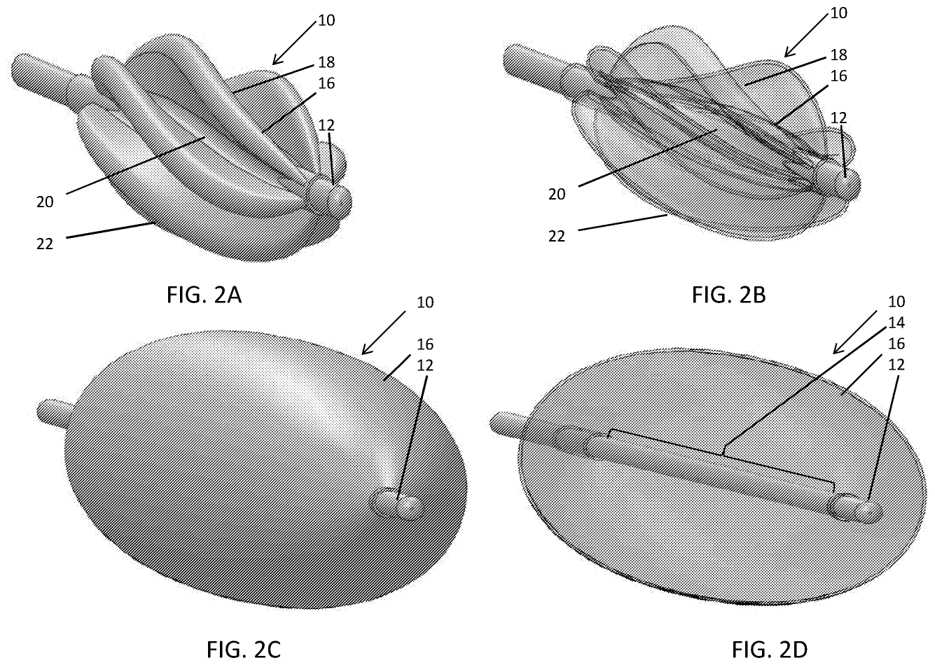

The present invention is detailed with respect to FIGS. 1A, 1B, 1C, 1D, 2A, 2B, 2C, and 2D where like numerals used in various drawings have a common meaning ascribed thereof, in which a blood pump is shown generally at 10 and includes a shaft 12 with at least one aperture 14 encompassed only within an inflatable membrane 16. The membrane includes a series of spiral pleats 18. The number of pleats ranges from 3 to 24. In some inventive embodiments, the number of pleats is between 4 and 12. In still other embodiments the pleats are uniform in size to define rotation symmetry around the central shaft 12, Cₙ, where n is an integer value of between 3 and 24. While the pleats 18 as depicted are defined by a single crease 20 with a radius of curvature that extends from the apex of a lobe 22 into proximity to the shaft 12. It is appreciated that creases may terminate between 10 and 70% distance between an apex of a lobe and the shaft. It is appreciated that a sharp crease may be a localized point of weakness of mechanical failure and in some inventive embodiments, a crease is formed to have a radius of curvature so as to avoid isoclinal walls, where the limbs of the fold are parallel, and instead spread the forces of pleating across a larger area with a tight fold with interlimb angles of up to 30 degrees and close folds with interlimb angles of between 30 and 70 degrees. It is appreciated that multiple creases between adjacent lobes servers to limit the inflection.

While the shaft is depicted in the drawings as being central and concentric with the membrane 16, it is appreciated the shaft may be curved or displaced so as no longer being concentric with the membrane. In an extreme example of asymmetry, the shaft lies along the membrane on one side and the membrane inflates circularly from this largely fixed edge. The depth of the pleats being circularly asymmetric as a result.

While it is appreciated that dimensions of a subject aorta dictate the scale of the device 10, a typical fully inflated diameter is from 12 to 25 millimeter (mm), with the embodiment depicted in FIG. 1C having an inflated diameter of 20 mm. While FIG. 1C shows a seemingly featureless surface, in some inventive embodiments the balloon 10 is inflated to a maximal extent in which shallow traces of the creases 20 are visible to the unaided, normal human eye. Thus, the minimum depth of a crease in uninflated form is anywhere between 0 and 100% of the final radius of the crease in a fully inflated state. In still other inventive embodiments, the minimum crease depth is 10% of the final radius in a fully inflated state.

The shaft 12 has a diameter dictated by anatomy; a typical shaft diameter is from 0.3 to 2.5 millimeter (mm), with embodiment depicted in the drawings having a diameter of 1 mm. A shaft 12 is formed from a variety of implant approved polymeric materials illustratively including polyurethanes, silicones, and perfluoropolymers. An attribute of an inventive membrane is that it is non-elastomeric in having a reversible extension of less than 10%. The shaft 12 is placed in fluid communication with a gas supplied for pulsatile pressurization with a timing designed to bolster the effective cardiac performance of a recipient's heart, typically in some form of failure that yield subnormal ejection characteristics. It is further appreciated that a shaft 12 with a degree of flexibility facilitates minimally invasive aortic placement. In still other embodiments the shaft 12 is formed of a material that retains an anatomically complaint shape to match the contours of a surrounding aorta. This can be accomplished with resort to a shape retaining surgically implantable polymer or metal, or through the inclusion of a malleable metal wire therein that will retain an assumed curvature. It is appreciated that the curvature can be implanted pre-surgically or during implantation.

The manifold apertures as depicted in the accompanying drawings are shown as a series of evenly spaced, like sized apertures. It is appreciated that gas flow simulations depending of the specifics of a particular inventive device, inflating gas characteristics, and pressurization profiles can also yield apertures with holes that increase in size distal from the pressure source to account for the drop-in pressure at a given aperture due to the gas transit through more proximal apertures along the manifold. Alternatively, like sized apertures vary in spacing to account for a pressure drop associated with more proximal apertures. Additionally, it is further appreciated that holes need not be circular in shape, with elongated slits and other simple geometric shapes also being operative herein, alone or in combination with circular or slit-shaped apertures. Well-defined control of the inflation cycle, leads to greater pressure assist to the adjacent implanted heart.

In some inventive embodiments, the membrane is textured and in still other embodiments, the membrane is integrally textured, meaning that a surface treatment is applied to the membrane material to create a textured surface amenable to biologic lining formation thereon. The properties of a fold-free textured membrane formed of polyurethane for use in a blood contacting surface are detailed in M. J. Menconi et al., J. of Cellular Biochem., 57:557-573 (1995). While not intending to be limited to a specific theory, the textured membrane is used to achieve at least one of the following objectives of: promote natural growth of a biologic lining on the surface of the in-dwelling pump to reduce the need for anticoagulation and the risk of thromboembolic events; promote washing of the surface to minimize stasis and thrombus formation; minimize strain on the membrane; minimize elongation radially and longitudinally to avoid fatigue of the membrane; minimize stretching and stress distribution along a balloon embodiment; promote a sweeping effect through the channels in the non-expanded state to wash the surface; or a combination thereof.

Thus, when a textured polyurethane is exposed to blood, the integrally textured membrane, such as one formed of polyurethane, is believed to develop a biofilm that in turn has puripotent cells attach thereto. These cells then flatten and take on the appearance and function of epithelial cells. In some inventive embodiments, the textured surface has an immuno-isolation coating on the membrane.

The thickness of the membrane 16 is dependent on factors such as the material from which is formed, the depth of any integral texturing, membrane size, and the kinetic pressure cycle. A typical membrane thickness is between 0.001 and 1 mm.

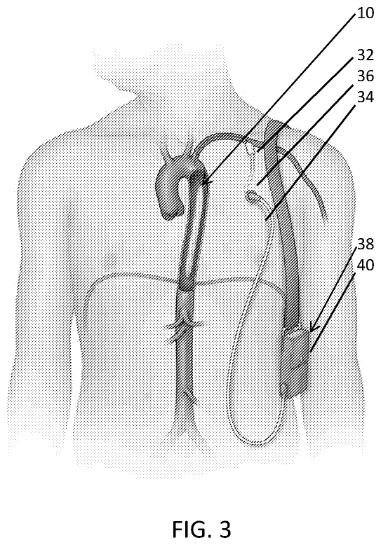

FIG. 3 is a schematic of a balloon 10 implanted in a patient with communication for a power or actuating connection 32 via conduit 34. A percutaneous access device (PAD) 36 extends through the skin surface layers (SL) illustratively including the epidermis, dermis, and subcutaneous tissue provides for a semi-permanent connection to an external fluid drive system and controller 38. As is described in greater detail in the prior patents incorporated herein by reference in their entirety, a conduit 34 can be led from the implanted balloon 10 to a percutaneous access device 36 implanted and projecting through a patient's skin.

The percutaneous access device 36 allows the gas communication tube and electrical leads shown generally as the conduit 34 as needed for sensors or other operational aspects, to be operatively connected to or disconnected from an external fluid drive system and controller, shown generally at 38. In operation, the balloon 10 or multiple such balloons are each independently cyclically inflated and deflated with a pressurized fluid with a synchronicity relative to the patient heart to increase effective cardiac output. Preferably, the synchronous cyclical inflation and deflation are based on a set of programmable patient parameters relating to heart function. The fluid driver 40 may supply an inflation fluid as either a gas or a liquid to expand the balloon 10 causing it to move form a deflated state to an inflated state with a timing to increase effective cardiac output. It is appreciated that gases other than air are operative with the present invention to induce pump inflation. These gases illustratively include helium, nitrogen, argon, and mixtures thereof. While these gases have lower viscosities than air, such gases necessitate tethering the recipient of an inventive blood pump implant to a compressed gas tank thereby reducing the mobility of the recipient. In a specific embodiment, a tracer may optionally be added to the fluid to detect a compromised membrane. Other fluids such as saline or other hydraulic fluids can serve to actuate the pumping chamber; optionally, a tracer substance such as indocyanine green or fluorescein can be included in the hydraulic liquid for detection of leaks from the pumping chamber.

Optionally, feedback sensors are provided for the operation of an inventive blood pump 10. Such sensors illustratively include a pressure transducer, an accelerometer, a strain gauge, an electrode, and species specific sensors such as pH, oxygen, creatine, nitric oxide or MEMS versions thereof. The output of such a sensor being transmitted as an electrical or optical signal to monitoring and regulatory equipment exterior to the body of the recipient.

Embodiments of the inventive cardiac pump alone or a plurality of such pumps in the aggregate displaces from about 20 to 70 cubic centimeters of blood upon inflation; each alone or collectively when several chambers are implanted and operating collectively. In a particular inventive embodiment, 50 to 70 cubic centimeters of blood are displaced per heartbeat by the present invention so as to allow an individual having an inventive pump implanted an active lifestyle. In still other embodiments, 60 to 65 cubic centimeters of blood per patient heartbeat by the present invention. The long axis of the shaft being aligned along the long axis of the aorta. Additional details regarding suitable control programs and methods of operation adaptable for use with the present invention can be obtained from U.S. Pat. Nos. 5,833,619, 5,904,666, 6,042,532, 6,132,363 and 6,511,412, all of which are incorporated by reference herein in their entireties.

The foregoing description is illustrative of particular embodiments of the invention, but is not meant to be a limitation upon the practice thereof. The following claims, including all equivalents thereof, are intended to define the scope of the invention.

1. An intra-aortic spiral balloon pump comprising:
a shaft having at least one aperture; and
an inflatable membrane encompassing said at least one aperture, said inflatable membrane comprising a plurality of spiral pleats about said shaft.

2. The pump of claim 1 wherein said plurality of pleats is from 3 to 24 pleats.

3. The pump of claim 1 wherein said plurality of pleats is from 4 to 12 pleats.

4. The pump of any one of claims 1 to 3 wherein said plurality of pleats define rotation symmetry, Cₙ, where n is an integer value of between 3 and 24 around said shaft.

5. The pump of claim 1 wherein two adjacent pleats of said plurality of pleats are separated by a single crease having a radius of curvature.

6. The pump of claim 5 wherein said crease extends from an apex of a lobe in said inflatable membrane into proximity to said shaft.

7. The pump of any one of claims 1 to 3 wherein said inflatable membrane has an exterior texture.

8. The pump of claim 7 wherein said inflatable membrane has an exterior texture that is integral to said inflatable membrane.

9. The pump of any one of claims 1 to 3 wherein said at least one aperture is a plurality of apertures.

10. The pump of claim 9 wherein said plurality of apertures are uniformly spaced.

11. The pump of claim 9 wherein said plurality of apertures vary in area as a function of length along said shaft.

12. The pump of any one of claims 1 to 3 wherein said membrane is formed of polyurethane.

13. A cardiac assist device comprising:
a pump of claim of 1;
a drive line in fluid communication with said pump; and
an external drive unit or fluid supply in fluid communication with said drive line.

14. The cardiac assist device of claim 13 further comprising a second pump of any one of claims 1 to 3.

15. The cardiac assist device of any one of claim 13 or 14 wherein said external drive unit further comprises a pump modifying a pressure of fluid in said inflatable cardiac pumping chamber with a periodicity to aid in blood movement through a subject aorta.

16. The cardiac assist device of any one of claim 13 or 14 further comprising a percutaneous access device intermediate between said drive line and said external drive unit or said fluid supply.

17. The cardiac assist device of any one of claim 13 or 14 further comprising an immuno-isolation coating on said membrane.
